(19) United States
(12) Patent Application Publication
(10) Pub. No.: US 2015/0300867 A1
(43) Pub. Date: Oct. 22, 2015

(54) DEVICE WITH FUNCTION FOR MEASURING THE VOLUMETRIC CHANGES IN A SUBSTANCE

(71) Applicants: CEMENTOS ARGOS S.A., Medellin (CO); UNIVERSIDAD EAFIT, Medellin (CO)

(72) Inventors: Maria F. DIAZ, Envigado (CO); Juan M. JARAMILLO, Medellin (CO); Carlos G. CORREA, Medellin (CO)

(21) Appl. No.: 14/646,714

(22) PCT Filed: Nov. 22, 2012
(86) PCT No.: PCT/IB2012/056605

§ 371 Date: May 21, 2015

(51) Int. Cl. G01F 23/292; G01N 33/38; G01F 22/00

(52) U.S. Cl. CPC G01F 23/292; G01F 22/00; G01N 33/383

(57) ABSTRACT

This invention relates to a device with function for measuring volumetric changes in a substance. In particular, the device with function for measuring volumetric changes in a substance comprises a sensor that detects the amount of light intensity produced by a light source aligned with said sensor when passing through a column of two liquids which are immiscible among each other contained in a transparent tube, wherein the sensor and the aligned light source are mounted on an arm that travels on a vertically arranged rail, wherein the arm is attached to a strap which acts by means of a first and a second pulley causing the movement of said arm, wherein the movement of said pulleys comes from a motor and a gearbox and the entire mobility system is mounted on a frame. The device further comprises data acquisition means which record the change of light intensity and control the arm movement.

### TECHNICAL FIELD

This invention relates to a device with function for measuring volumetric changes in a substance, comprising a sensor that detects the amount of light intensity produced by a light source aligned with said sensor, when passing through a column of two liquids which are immiscible among each other contained in a transparent tube, wherein the sensor and the aligned light source are mounted on an arm that travels on a vertically arranged rail, wherein the arm is attached to a strap which acts by means of a first and a second pulley causing the movement of said arm, wherein the movement of said pulleys comes from a motor and a gearbox and the entire mobility system is mounted on a frame. The device further comprises data acquisition means which record the change of light intensity and control the arm movement.

### PRIOR ART

According to the prior art, there are disclosed several applications of the device; among them, the indirect monitoring of the chemical shrinkage of concrete associated with the hydration speed of said material, as indicated in the ASTM C1608 standard. As the concrete hydration products use less volume than the initial materials before reacting (concrete and water), the speed of said volumetric change in materials indicates the degree of chemical shrinkage the concrete has. Similarly, the art discloses some measuring instruments backgrounds in this regard, such as that disclosed in the United States document U.S. Pat. No. 6,986,279 to Lockwood et al., which discloses a device for determining the liquid absorption of an aggregate which comprises providing a stirring system, a vacuum source and a container; placing a sample of the aggregate in the container; adding enough liquid to the container so as to achieve calibration marking in the container; weighing the sample and liquid; mounting the container in the stirring device; connecting the vacuum source to the container; stirring the sample and the liquid with the stirring device; applying a vacuum to the sample and the liquid with the vacuum source, after the stirring and vacuum steps, adding enough liquid to the container so as to achieve again he calibration marking in the container, weighing again the sample and the liquid, and subtracting the initial weight of the sample and liquid of the final weight of the sample and liquid in order to determine the liquid absorption in the aggregate. The apparatus of the art complies with the need of measuring the concrete absorption but is of high-cost construction and does not monitor the sample during the entire process, obtaining intermediate data. Further, this prior art equipment is of difficult manufacturing, handling and transportation.

### DISCLOSURE OF THE INVENTION

### Technical Problem

The object of the present invention is to improve the shortcomings of the prior art. More particularly, the main object of the present invention is the measuring device which relates the volumetric changes of a substance depending on the systematic absorption.

A second object is that from the device of the invention, it is possible to determine the amount of a substance in a solution.

Another important object of the present invention is to create a de vice comprising a sensor aligned to a light source, supported on a frame and including means for performing a stroke of the sample.

The present invention meets these needs and provides other related advantages.

The novel features that are considered as the base of the invention are cited in the enclosed claims and the additional advantages thereof will be better understood by the following detailed description with the preferred embodiments and the proper reference to the drawings attached thereto.

### Solution to the Problem

Advantageous Effects of the Invention

### BRIEF DESCRIPTION OF THE DRAWINGS

In order to further clarify The invention and the advantages thereof compared with the known art, the possible illustrative and non limitative embodiments of the application of said invention are described below with the support of the drawings.

FIG. 1 shows a right side view of the device with function for measuring the volumetric changes in a substance of the present invention.

FIG. 2 shows a rear view of the device with function for measuring the volumetric changes in a substance of the present invention.

FIG. 3 shows a front view of the device with function for measuring the volumetric changes in a substance of the present invention.

FIG. 4 shows in detail the sample collection column of the device with function for measuring the volumetric changes in a substance of the present invention.

FIG. 5 shows a schematic view of the device with function for measuring the volumetric changes in a substance, including the interconnection with the systems of data recording and collection according to the present invention.

### BEST WAY OF CARRYING OUT THE INVENTION

### Method of Carrying Out the Invention

This invention relates to a device with function for measuring volumetric changes in a substance. Particularly, the device with function for measuring volumetric changes in a substance comprises a sensor aligned to a light source which allows for measuring the change of intensity.

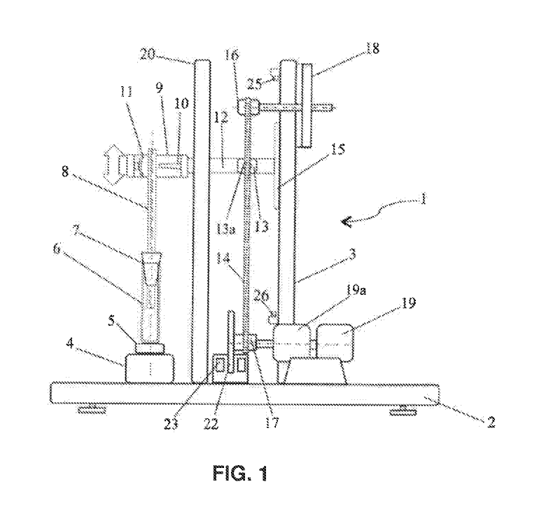

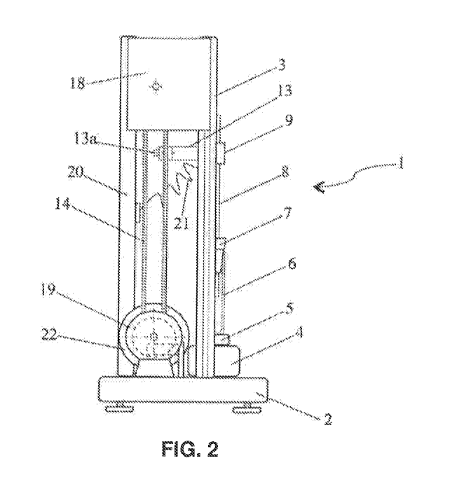

According to FIGS. 1, 2 and 3, the device 1 for measuring volumetric changes in a substance of invention, comprises a base frame 2 which receives a mast frame and a sample collection column 4. The sample collection column 4, in turn, comprises a receiving and positioning base 5 which supports a vessel or container 6 with top mouth opened to the environment and preferably made of glass. This vessel 6 includes a plug 7 which covers and closes said top mouth. The plug 7 further comprises a central through hole, wherein a transparent tube 8 is vertically provided and which is opened at its both ends, preferably made of glass and with graduation marks or reglette and which protrudes over the plug at least three folds the vessel 6 height, with a portion of the tube 8 entering between the vessel 6 towards the middle height of said vessel 6, traversing the plug 7. An elongated support 9 is provided aligned with the tube 8 protrusion, arranged perpendicularly with respect to the transparent tube 8, including a light source 10 and a sensor 11 facing each other, surrounding the tube 8.

In a preferred embodiment, an in an illustrative fashion, the light source 10 used for the present invention may comprise a DI650-2.5-3(5) reference laser, with a 650 nm wavelength, red colored visible spectrum, with 2 mm spot diameter and a 1.5 mrad divergence, having a 2.5 mW power output, manufactured by the company SHAANZI HUAKE OPTOELECTRONICS CO. LTD.

Likewise, the sensor 11 which allows for recording the light intensity of the light source 10 when it passes through a liquid means is an ODD-660 W reference diode from the company OPTO-DIODE CORP. In an illustrative but not limitative manner, for the present invention, the sensor 11 comprises an effective 0.62 mm² area and a response centered at 650 nm of 0.42 A/W.

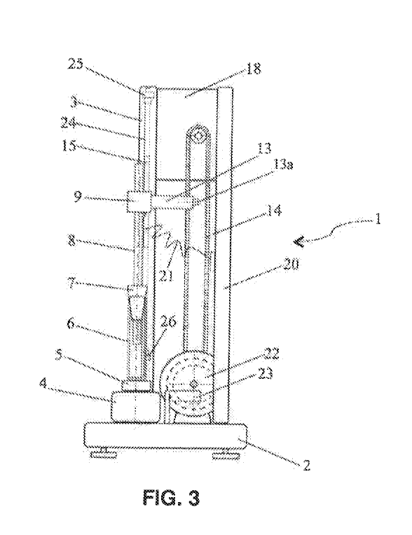

The support 9 is mounted at the end of an elongated arm 12 which protrudes horizontally; wherein said arm 12 comprises an extension 13 including a fixed connection 13a to a vertically arranged strap 14, wherein the extension 13 protrudes perpendicularly from he arm 12. To ensure the correct movement of the arm 12 towards the end opposed to the support 9, the arm 12 is attached to a lane 15 which travels along a rail 24 comprised by a channel which is vertically arranged along the mast 3 as shown in FIG. 3. The lane 15 ensures vertical movement of the arm 12 as shown in FIG. 1, so as to cause the support 9 including the light source 10 and the sensor 11 to travel the free length of the tube 8.

For the vertical movement of the arm 12, the strap 14 acts by means of connection to the top 16 and bottom 17 sets of pulleys. The top pulley axis 16 is supported by a plate 18 comprising an L-fold attached to the rear top end of the frame mast 3 as shown in FIGS. 1, 2 and 3. For its rotation, the bottom pulley 17 is connected to a motor 19 and a gearbox 19a, which allows for the mechanical movement of the device.

In preferred embodiments, as shown in FIGS. 1, 2 and 3, in order to attach the connecting cables to the support 9, the device of the invention comprises a cable mast 20 which arranges and positions the connection cable 21 towards the support 9 and connects the light source 10 and the sensor 11.

The control for the movements of the arm 12 comprises an encoder 22 comprising an infrared pair 23 provided adjacent to the encoder 22, wherein said encoder 22 is coupled to the protrusion of the bottom pulley 17 axis. Said encoder 22 along with the infrared pair 23, are located for detecting the rotation and encode same so as to control the moor 19 operation and its direction of rotation.

Thus, the motor 19 provides the movement for raising and lowering the arm 12, allowing for the support 9 to travel along the entire length of the tube 8.

In preferred embodiments of the invention, at the ends of the rail 24 attached to the mast 3, there are provided the top 25 and bottom 26 sensors such that when the lane 15 along with the arm 12 reach these points, the system immediately detects the position of the lane 15 and, in such case, stops the time and height recording and returns to the initial position (end of the run at the top of the rail support).

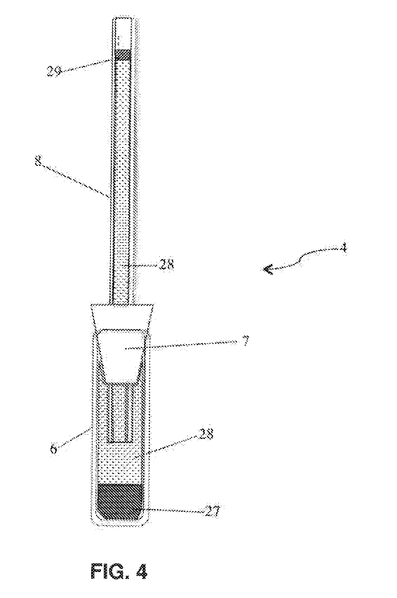

According to FIG. 4, for its use in tests or trials, the sample collection column 4 contains within the vessel 6 a first volume of the solid or liquid 27 which. will absorb a volume of liquid 28, a portion of which being provided within the vessel 6 and another portion within the tube 8, followed, in order to cover and close the environment, by a different colored and immiscible substance 29 of the liquid 28 to be absorbed. As the transparent tube 8 is opened on both sides, when introducing same in the vessel 6 with plug 7, the level of the liquid (e.g., water) contained in the vessel 6 will rise through the tube 8 up to a specified level. Subsequently, a small volume of an immiscible liquid (e.g., oil) and with an absorption rate significantly different from the first is added over the specified level.

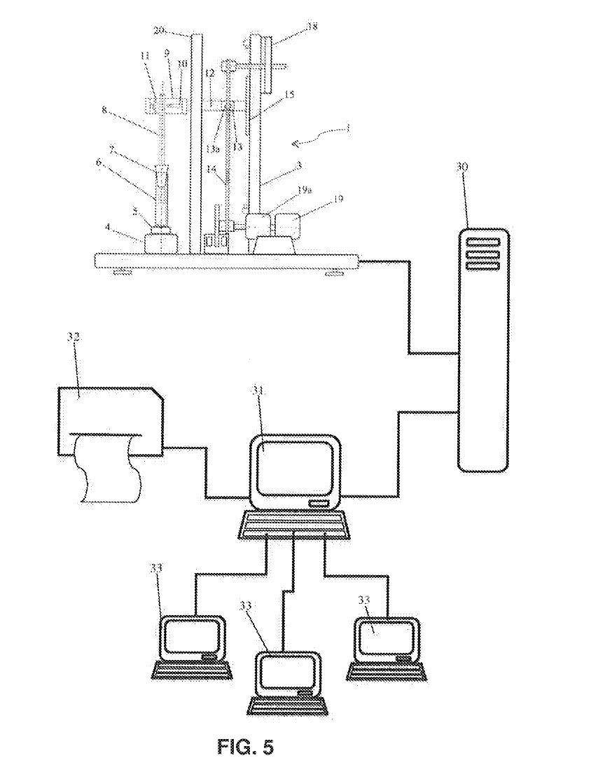

Based on FIG. 5, in order to determine the record of the data emitted from the support 9 including the light source 10 and the sensor 11 both for regulating the movement of the arm 12 as well as all the variables of the trial, electronic processing means 30 are provided for establishing, through a logic support, an order of sequences to be treated. Further, the equipment comprises a computerized terminal 31 for managing such data, printing printing means 32, and data transmission, if applicable, to remote terminals 33.

The device of the invention has been conceived such that the data are received by the software, the latter being responsible for interpreting the voltage values recorded by the sensor 11 and providing the information required by the for measuring and analysis. Then, the electronic processing means must perform the functions of recording the light intensity emitted by the light source 10 detected by the sensor 11; moving said light source and sensor along the tube 8; recording the amount of movement; and recording the time of change of means for later saving said data in an internal memory which is part of the electronic plate.

In order to perform these movement and recording functions, electronic support components such as a control unit which component is a micro-controller are provided, through which the commands are sent to the mechanical system wherein the motor 19 is started by relays.

The device of the invention further comprises a memory in which the data are stored.

Likewise, so as to ensure a correct leveling of the device 1 over the working surface, the base frame 2 may comprise levels along with height adjustable supports such as bolt-adjustable legs.

The entire system is controlled by the micro-controller, which receives instructions from a computer. After being started by the computer, the system is independent and collects the required information as per its programming.

According to FIGS. 1, 2 and 3, when the equipment is started, the arm 12 and the support 9 start descending and recording the light intensity crossing the air contained within the tube 8. The mechanical arm 12 starts moving vertically towards the base or bottom end of the tube 8 until it encounters the light intensity corresponding to the immiscible liquid (e.g., oil). The equipment records that height value and time equals zero as a baseline level. From this baseline level, the mechanical arm 12 descends to a specified height remaining fixed. At that point, the time starts to be recorded until the immiscible liquid (e.g., oil) reaches the specified height and the light intensity variation is produced when passing from the absorbed liquid (e.g., water) to the immiscible liquid (e.g., oil). When recording such time that the immiscible liquid took for reaching the specified height, the mechanical arm 12 once again travels vertically along the same specified distance. The height that the system records at this point is measured from the baseline level; thus, the traveled distance is equal to twice the specified height. In this new position it remains static until the immiscible liquid (e.g., oil) reaches the new height which is equal to twice the specified height. When this happens, a variation of intensity occurs when crossing the interface (absorbed liquid and immiscible liquid). When the immiscible liquid reaches the new height position, the device of the invention records the time elapsed from the baseline level until that time. Once the new height and time (cumulative) recorded, the arm travels again to the initial specified height. The height that the equipment records now is a third height and the system behaves as described above.

The device, through a data acquisition card in the electronic processing means, records the change of light intensity produced as a result of the movement of the liquid to he absorbed (e.g., water) contained in the tube since the solid or liquid absorbs some amount of volume thereof and along with the movement of the volume of the liquid to be absorbed, the movement of the different colored and immiscible substance (e.g., oil) which is in the top of the column is established. When the volume of the different colored and immiscible substance (e.g. oil) reaches the light beam that comes from the light source 10, through the sensor 11, the equipment records and saves the time and height in which this change of intensity occurred when the light passed from one means to another (e.g., from water to oil).

Similarly, from the device of the present invention, it is possible to determine the amount of a substance in a solution.

The preferred embodiments of he invention have been depicted solely by way of example. In this regard, it will be noted that the device with function for measuring the volumetric changes in a substance of the present invention, as well as the arrangements in its particular provision, may be chosen from a plurality of alternatives without departing from the spirit of the invention according to the following claims.

### Industrial Applicability

Sequence listing free text

1. A method for measuring the volumetric changes in a substance which is part of a sample provided within a vessel with opened top mouth, wherein the mouth is closed by a plug having a hole through which a transparent tube with opened ends and graduation mark passes, the tube having its bottom end inside the vessel and its top end outside the vessel, the vessel being filled with a translucent liquid which further fills a tube length until it forms a level of the translucent liquid, a light emitter/receptor set being comprised by a light emitter provided at one side of the tube and a light sensor at the opposite side of the tube, said light emitter and sensor being optically aligned, wherein the method comprises the step of:
Providing an immiscible marker with said translucent liquid and which has opacity over said level of translucent liquid,
Providing said light emitter/receptor set along the tube and over said marker,
Moving said light emitter/receptor set downstream until said marker interposes between said light emitter and said light sensor, thereby determining a variation in the intensity of the light traversing the tube,
Defining at that point a zero “0” time and an initial position or baseline position,
Moving said light emitter/receptor set downstream along a specified distance until a first position, and recording at that point a height value,
Waiting for said marker, in a downstream direction, to interpose between said light emitter and said light sensor in said first position, thereby determining a new variation in the intensity of the light reaching the light sensor,
Recording said height value and the time the marker took for interposing between the light emitter and the sensor;
Moving said light emitter/receptor set downstream until a second position, and recording at that point the new height value,
Waiting for said marker, in a downstream direction, to interpose between said light emitter and said light sensor, thereby determining a new variation in the intensity of the light reaching the light sensor,
Recording at that point said height value and the time the marker took for interposing between the light emitter and the sensor;
Moving said light emitter/receptor set downstream until a third position, and recording at that point a new height value,
Waiting for said marker, in a downstream direction, to interpose between said light emitter and said light sensor and, when the marker is not able to interpose between the emitter and the sensor in a specified time, establishing the second position as the last position;
Recording the distance between the initial position and the last position, and
Recording the time elapsed between the time zero “0” and the time accumulated until the last position.

2. A method according to claim 1, wherein said translucent liquid is water and said marker is a colored immiscible liquid.

3. A method according to claim 2, wherein said colored immiscible liquid is oil.

4. A device for measuring, by means of the method of claim 1, the volumetric changes in a substance which is part of a sample, the device comprising a base frame which receives a mast frame and a sample collection column; wherein the sample collection column in turn comprises a receiving and placement base supporting a vessel or container with top mouth opened to the environment and made of transparent material; wherein said vessel comprises a plug which covers and closes said top mouth; wherein the plug further comprises a central through hole (with no reference numeral), wherein a transparent tube is provided opened at its both ends, with graduation marks or reglette and which protrudes over the plug at least three folds the vessel height, with a portion of the tube entering between the vessel towards the middle height of said vessel, traversing the plug; the tube and the vessel containing a liquid defining a top level where a marker immiscible with the liquid and with opacity floats, wherein an elongated support is provided aligned with the tube protrusion, arranged perpendicularly with respect to the transparent tube, including a light source and a sensor facing each other, surrounding the tube; wherein the support is mounted at an end of an elongated arm which protrudes horizontally, wherein said arm comprises an extension including a fixed connection to a vertically arranged strap, wherein the extension protrudes perpendicularly from the arm; and wherein the arm, towards the end opposed to the support, is attached to a lane which travels along a rail comprised by a channel which is vertically arranged along the mast.

5. The device according to claim 4, wherein the strap acts by means of a connection to a top and bottom set of pulleys.

6. The device according to claim 5, wherein the top pulley axis is supported by a plate comprising an L-fold attached to the rear top end of the frame mast.

7. The device according to claim 6, wherein the bottom pulley is connected to a motor and a gearbox.

8. The device according to claim 7, comprising a cable mast which arranges and positions the connection cable towards the support and connects the light source and the sensor.

9. The device according to claim 8, wherein the control for the movements of the arm comprises an encoder comprising an infrared pair provided adjacent to the encoder, wherein said encoder is coupled to the protrusion of the bottom pulley axis.

10. The device according to claim 9, wherein at the ends of the rail attached to the mast, there are provided the top and bottom sensors for detecting the position of the lane.

11. The device according to claim 10, wherein the recording of the data emitted by the sensor, based on which the movement of the arm is regulated, is provided with electronic processing means for establishing, through a logic support, an order of sequences to be treated.

12. The device according to claim 11, comprising a computerized terminal for managing such data; printing means; and remote terminals.

13. The device according to claim 12, comprising a micro-controller which commands the mechanical system; and wherein the motor is started by relays.

14. The device according to claim 13, comprising a data storage memory.

15. The device according to claim 14, wherein 15 the base frame may comprises levels and height adjustable supports.
